(19) United States
(12) Patent Application Publication
(10) Pub. No.: US 2010/0093544 A1
(43) Pub. Date: Apr. 15, 2010

(54) ADJUVANT COMPOSITION

(75) Inventors: Darren J. Park, Western Australia (AU); John R. Horley, Western Australia (AU)

Correspondence Address: NIXON & VANDERHYE, PC, 901 NORTH GLEBE ROAD, 11TH FLOOR, ARLINGTON, VA, 22203, US

(73) Assignee: OPAL AUSTRALASIA PTY LTD., Kwinana, Western Australia (AU)

(21) Appl. No.: 12/520,633

(22) PCT Filed: Dec. 24, 2007
(86) PCT No.: PCT/AU2007/002004

§ 371 Date: Jun. 22, 2009

(30) Foreign Application Priority Data
Dec. 22, 2006 (AU) 2006907192

(51) Int. Cl. A01N 25/12; A61K 9/14; A61K 47/02

(52) U.S. Cl. 504/367; 514/769; 424/489

(57) ABSTRACT

A solid substantially water-soluble adjuvant composition 32 comprising a surfactant 24 and a particulate nitrogen-based plant nutrient 12. The surfactant 24 is supported on the particulate plant nutrient 12 which has been pre-milled to a suitable particle size. The surfactant 24 is optimised to a viscosity of less than about 100 mPa·s at 20 degrees Celsius. The invention may further comprise the addition of acidifier 26 to the composition, the viscosity of the acidifier and the surfactant having been optimised to produce the desired solid water-soluble adjuvant composition 32. A method 10 is also described which involves the milling 14 of a particulate nitrogen-based plant nutrient 12 which is then mixed with surfactant 24 and/or acidifier 26. The viscosity of the surfactant and/or acidifier are chosen or adjusted to appropriate levels to produce an optimum adjuvant composition.

### FIELD OF THE INVENTION

The present invention relates to an adjuvant chemical composition, and a method of producing an adjuvant chemical composition.

### BACKGROUND TO THE INVENTION

Agrochemicals such as pesticides and herbicides are widely used in agricultural applications for the protection of crops. Adjuvant compositions are often combined with crop protection agents such as herbicides in a spray tank mixture to increase the efficacy of the herbicide. Adjuvants can generally be described as substances that modify or enhance the effect of other agents, in this case the other agents being herbicides and/or pesticides.

The types of substances that are used as adjuvants include ammonium salts such as ammonium sulfate, as well as other plant nutrients or fertilisers. Ammonium salts are believed to minimise the negative effects of hard water on herbicidal performance and to provide nutrition to the crop plant which in turn improves the herbicidal efficacy. Wetting agents, surfactants, spray drift retardants, oils, and water conditioners (such as acidifiers) are also used as adjuvants. Wetting agents or surfactants lower the surface tension of the herbicide and water spray tank mixture, and assist in improving the leaf coverage of the herbicide, and penetration through the leaf cuticle. Acidifiers are used to reduce the spray mixture pH. Many pesticides display improved activity when applied in weakly acidic spray mixtures (below pH 6) but many water supplies are alkaline (above pH 7). Lowering the pH improves penetration of the pesticide and reduces decomposition of the pesticide in the spray mixture.

The herbicide glyphosate is a non-selective herbicide and is used extensively in agricultural, industrial and household applications to control weeds.

Ammonium sulfate is widely used as a fertiliser source of nitrogen and sulfur. As an herbicide adjuvant, ammonium sulfate is typically added to a spray tank solution or dispersion of crop protection agent at the point of use. When used as an adjuvant, ammonium sulfate can condition hard water by interacting with dissolved minerals such as calcium and magnesium. In the case of herbicides such as glyphosate, this enhances activity by preventing these minerals from interacting with the dissolved glyphosate. If the minerals do interact with the dissolved glyphosate, plant uptake of the glyphosate is reduced, thereby leading to antagonism. In addition, the presence of dissolved ammonium from the ammonium sulfate improves the solubility of the glyphosate and is believed to enhance absorption by the target weeds.

Commercially available ammonium sulfate formulations used for this purpose may be coarse crystalline powders, granules or concentrated solutions in water. There are several disadvantages associated with the use of these conventional ammonium sulfate formulations. Coarse crystalline powders have the disadvantage that dissolution into water is relatively slow and recirculation of the hard crystals have been implicated in premature failure of pumping systems. Milling of crystalline ammonium sulfate to form a finer product with improved water dissolution characteristics would not normally be considered in usual practice since the hydroscopic nature of ammonium sulfate causes mill blockages to occur. Finely divided ammonium sulfate has a strong propensity to rapidly absorb water from the air causing hardening that makes the product more difficult to use than the original coarse crystals. Fine crystalline powder formulations must have an anti-caking agent added prior to milling to reduce hardening. In addition, these fine crystalline powder formulations are very dusty, making handling difficult and representing a potential health hazard.

Liquid formulations comprising ammonium sulfate are limited by the solubility of ammonium sulfate in water (practical limit is about 500 g/L) resulting in the need to transport and handle large volumes of water. These solutions require a sturdy (usually plastic) container which adds expense and generates disposal issues.

Surface active agents (or surfactants) represent a class of (usually organic) compounds which can adsorb at phase interfaces and thereby reduce interfacial tension. Surfactants have wide application in crop protection as wetting, penetrating, dispersing and stabilising agents. As adjuvants, surfactants are commonly added to a spray tank solution, or to a dispersion of crop protection agents, to enhance activity by promoting wetting and penetration of the crop protection agent. Most commercially available surfactant formulations are liquids comprising a single surfactant compound, or simple blends.

As for conventional ammonium sulfate formulations, due to their composition conventional wetter adjuvant formulations have the disadvantage of requiring a sturdy (usually plastic) container which adds expense and generates disposal issues.

Acidifiers are a class of water conditioner added to a spray tank solution or to a dispersion of crop protection agents such as a herbicide or pesticide, to decrease the spray water pH to below pH 6. Many water supplies used to make spray mixtures are alkaline (higher than pH 7). The absorption of acid type herbicides such as glyphosate is improved under acidic conditions and this enhances activity. In addition, many pesticides degrade more rapidly in the spray solution under alkaline conditions, which leads to a decrease in activity, and accordingly acidifiers are used to assist with this problem. Commercially available acidifier adjuvants are usually solutions of an acid in water.

Since ammonium sulphate, surfactants and acidifiers (as adjuvants) each perform different functions, it is desirable to have a single formulation that combines these three products with their associated functions. Such a formulation has the advantage of replacing three separately added products with one, with the potential for considerable savings in handling and packaging. Combined formulations of ammonium sulfate and surfactants have been previously developed, but these have focused on liquid based formulations that have several disadvantages including the following:

(1) The practical solubility limit of ammonium sulfate in water is about 500 g/litre, limiting the strength of the formulation.

(2) The presence of concentrated, dissolved ammonium sulfate inhibits the solubility of many surfactants thus limiting the type and amount of surfactant that may be used in the formulation.

(3) The added water in the formulation greatly increases the volume of the product. This has a negative impact on packaging, transportation and handling costs.

A simple admixture of ammonium sulfate together with liquid surfactants or acidifiers of the type commonly used as adjuvants, at typical usage ratios, produces a heavy wet semi-slurry which is difficult to handle. It also adheres excessively to packaging making efficient use difficult. It also tends to form a separate pool of liquid at the bottom of the container, and in many cases displays poor solubility properties in water despite all components having high water solubility.

Rapid and complete mixing of an adjuvant composition in a spray tank mixture is critical to avoid degradation or decrease in performance. Excessive loss of the liquid wetting and/or acidifiying component can occur by adherence on packaging if the mixture is a slurry, and wetting and/or acidifying performance is degraded as a result. Rapid dissolvability in water is important as a homogeneous spray tank solution of crop protection products is vital to ensure uniform and predictable activity. If dissolution is slow, formation of a persistent sludge layer on the bottom of the spray tank may occur.

Thus there is a need to produce a combined adjuvant formulation which retains the advantages of the separate formulations but which at least partly alleviates the disadvantages of the present combined formulations.

It is an object of the present invention to provide a single adjuvant composition which combines the functions of the separate constituents but which minimises, reduces or avoids the usual problems of a liquid material, a dense slurry material, dust particles, and packaging and storage problems associated with prior art formulations.

References to prior art in this specification are provided for illustrative purposes only and are not to be taken as an admission that such prior art is part of the common general knowledge in Australia or elsewhere.

### SUMMARY OF THE INVENTION

The present invention provides a solid substantially water-soluble adjuvant composition comprising a surfactant and a particulate nitrogen-based plant nutrient, the surfactant being supported on the particulate plant nutrient.

Preferably the solid substantially water-soluble adjuvant composition is used in an agricultural chemical application. Preferably the solid substantially water-soluble adjuvant composition is used with a pesticide. More preferably the solid substantially water-soluble adjuvant composition is used with a herbicide, such as by mixing with the herbicide in a spray tank mixture prior to application. Typically the herbicide is glyphosate but the invention may also have application with other herbicides and other pesticides.

Preferably the particulate nitrogen-based plant nutrient is a nitrogen-based fertiliser. Preferably the nitrogen-based fertiliser is an ammonium compound. More preferably the nitrogen-based fertiliser is ammonium sulfate. Other ammonium based salts may also be used such as ammonium phosphate. Alternatively the nitrogen-based fertiliser may be urea.

Preferably the solid substantially water-soluble adjuvant composition is flowable. Preferably the solid substantially water-soluble adjuvant composition is granular. The granules preferably have a particle size of less than 3000 microns, and more preferably less than 1000 microns.

In a preferred form the particulate nitrogen-based plant nutrient has been milled to a particle size of less than 1000 microns. More preferably the particulate nitrogen-based plant nutrient has been milled to a particle size distribution of 80% less than 1000 microns.

Preferably the particulate nitrogen-based plant nutrient has been milled to a particle size of less than 500 microns and more preferably to a particle size of less than 250 microns. In a preferred form of the invention, the particulate nitrogen-based plant nutrient has been milled to a particle size distribution of 70% less than 500 microns and 30% less than 250 microns.

The pre-processing (milling) of coarse crystalline “as received” nitrogen based plant nutrient to produce a suitable particle size distribution prior to addition of the liquid component(s) (surfactant) is an important element of this invention. To enable this processing to occur readily, a small amount of anti-caking agent (less than 0.3%) can be blended with the particulate nitrogen-based plant nutrient prior to milling. This is less than the amount required to reduce hardening of the milled material on standing and is only needed to enable the size reduction step to occur without mill blockage and to assist with handling of the milled material prior to addition of the liquid component. However it should be noted that added anti-caking agent is not required for formulation functionality.

Preferably the solid substantially water-soluble adjuvant composition further comprises an acidifier. The acidifier is preferably supported on the particulate plant nutrient. Preferably the acidifier is supported on the particulate nitrogen-based plant nutrient by adsorption and/or absorption. Whilst not wishing to be limited by theory, it is believed that the acidifier may be supported on the particulate nitrogen-based plant nutrient by a combination of adsorption and/or absorption on the particle surfaces, adsorption and/or absorption into the pores or holes, and chemisorption.

The acidifier may take various forms and may comprise one or more compounds selected from the group consisting of inorganic liquid acids, inorganic solid acids, organic liquid acids, or organic solid acids. Preferably the acidifier is water soluble. Preferably the acidifier comprises one or more compounds selected from the group consisting of acetic acid, propionic acid, phosphoric acid, and citric acid.

The acidifier is preferably a liquid or a blend of liquids. The acidifier preferably has a viscosity of less than about 400 mPa·s at 20 degrees Celsius. More preferably the viscosity is less than about 100 mPa·s at 20 degrees Celsius. In a preferred form of the invention, the viscosity of the acidifier is less than 70 mPa·s at 20 degrees Celsius.

The surfactant may take various forms and may be a non-ionic, anionic or cationic surfactant. Preferably the surfactant comprises one or more compounds selected from the group consisting of alkyl phenol ethoxylates, alkyl phenol alkoxylates, alcohol exthoxylates, alcohol alkoxylates, aminoethoxylates or modified alkylpolysiloxane surfactants, but is not limited to these. Preferably the surfactant is a commercially available surfactant and may be selected from the group consisting of one or more of the “Teric” (registered trade mark of Huntsman Corp) range of surfactants including “Teric N8”, “Teric 9A6” or “Teric BL8”.

Preferably the surfactant is present in the solid substantially water-soluble adjuvant composition in an amount of less than 25% of the composition. More preferably the surfactant is present in an amount of less than 20% of the composition. In a preferred form of the invention, the surfactant is present in an amount of 10% to 15% of the composition. In a further form of the invention, the surfactant is present in an amount of less than 10% of the composition. At these levels, the ratio of surfactant: nitrogen-based plant nutrient is between about 1 and 2 parts surfactant to about between 9 and 8 parts nitrogen-based plant nutrient (ammonium sulfate), which is a biologically useful ratio.

The surfactant is preferably a liquid or a blend of liquids. The surfactant preferably has a viscosity of less than about 400 mPa·s at 20 degrees Celsius. More preferably the viscosity is less than about 100 mPa·s at 20 degrees Celsius. In a preferred form of the invention, the viscosity is less than 70 mPa·s at 20 degrees Celsius.

Preferably the surfactant is supported on the particulate nitrogen-based plant nutrient by adsorption and/or absorption. Whilst not wishing to be limited by theory, it is believed that the surfactant may be supported on the particulate nitrogen-based plant nutrient by a combination of adsorption and/or absorption on the particle surfaces, adsorption and/or absorption into the pores or holes, and chemisorption.

In a further form of the invention, the present invention provides a solid substantially water-soluble adjuvant composition comprising an acidifier and a particulate nitrogen-based plant nutrient, the acidifier being supported on the particulate plant nutrient.

Preferably the particulate nitrogen-based plant nutrient is a nitrogen-based fertiliser. Preferably the particulate nitrogen-based plant nutrient is an ammonium compound. More preferably the particulate nitrogen-based fertiliser is ammonium sulfate. Other ammonium based salts may also be used such as ammonium phosphate. Alternatively the nitrogen-based fertiliser may be urea.

The acidifier may take various forms and may comprise one or more compounds selected from the group consisting of inorganic liquid acids, inorganic solid acids, organic liquid acids, or organic solid acids. Preferably the acidifier is water soluble. Preferably the acidifier comprises one or more compounds selected from the group consisting of acetic acid, propionic acid, phosphoric acid, and citric acid.

Preferably the acidifier is present in the solid substantially water-soluble adjuvant composition in an amount of less than 25% of the composition. More preferably the acidifier is present in an amount of less than 20% of the composition. In a preferred form of the invention, the acidifier is present in an amount of 10% to 15% of the composition. In a further form of the invention, the acidifier is present in an amount of less than 10% of the composition.

The acidifier is preferably a liquid or a blend of liquids. The acidifier preferably has a viscosity of less than about 400 mPa·s at 20 degrees Celsius. More preferably the viscosity is less than about 100 mPa·s at 20 degrees Celsius. In a preferred form of the invention, the viscosity of the acidifier is less than 70 mPa·s at 20 degrees Celsius.

The solid substantially water-soluble adjuvant composition may further comprise other additives such as antifoaming agents, diluents and inert diluents.

The solid substantially water-soluble adjuvant composition is preferably in a granular or particulate form and is free-flowing and relatively dust free.

The present invention also provides a method for the preparation of a solid substantially water-soluble adjuvant composition comprising:
(i) milling a particulate nitrogen-based plant nutrient; and
(ii) mixing a surfactant with the milled nitrogen-based plant nutrient so that the surfactant is supported on the milled nitrogen-based plant nutrient.

In a preferred form the particulate nitrogen-based plant nutrient is milled to a particle size of less than 1000 microns. More preferably the particulate nitrogen-based plant nutrient is milled to a particle size distribution of 80% less than 1000 microns.

Preferably the particulate nitrogen-based plant nutrient is milled to a particle size of less than 500 microns and more preferably to a particle size of less than 250 microns. In a preferred form of the invention, the particulate nitrogen-based plant nutrient is milled to a particle size distribution of 70% less than 500 microns and 30% less than 250 microns.

Preferably the particulate nitrogen-based plant nutrient is a nitrogen-based fertiliser. Preferably the particulate nitrogen-based fertiliser is ammonium sulfate. Alternatively the nitrogen-based fertiliser may be urea.

Preferably the solid substantially water-soluble adjuvant composition is flowable and granular.

Preferably the method comprises a further step of adding an acidifier to the milled nitrogen-based plant nutrient so that the acidifier is supported on the particulate plant nutrient. The acidifier may take various forms and may comprise one or more compounds selected from the group consisting of inorganic liquid acids, inorganic solid acids, organic liquid acids, or organic solid acids. Preferably the acidifier is water soluble. Preferably the acidifier comprises one or more compounds selected from the group consisting of acetic acid, propionic acid, phosphoric acid, and citric acid.

Preferably the acidifier and the surfactant are mixed to form a combined adjuvant solution prior to mixing with the milled nitrogen-based plant nutrient. The combined adjuvant solution preferably has a viscosity of less than about 400 mPa·s at 20 degrees Celsius. More preferably the viscosity is less than about 100 mPa·s at 20 degrees Celsius. In a preferred form of the invention, the viscosity of the combined adjuvant solution is less than about 70 mPa·s at 20 degrees Celsius. By adjusting the viscosity of the combined adjuvant solution to suitable levels, it has been found that the properties of the solid substantially water-soluble adjuvant composition may be optimised as appropriate for the invention.

The method of the invention may also comprise the further addition of a free flowing agent to assist in avoiding mill blockages. A free flowing agent may be one or more of synthetic precipitated silicas, kaolins and attapulgites.

The surfactant may take various forms and may be a non-ionic, anionic or cationic surfactant. Preferably the surfactant comprises one or more compounds selected from the group consisting of alkyl phenol ethoxylates, alkyl phenol alkoxylates, alcohol exthoxylates, alcohol alkoxylates, aminoethoxylates or modified alkylpolysiloxane surfactants, but is not limited to these. Preferably the surfactant is a commercially available surfactant and may be selected from the group consisting of one or more of the “Teric” (registered trade mark of Huntsman Corp) range of surfactants including “Teric N8”, “Teric 9A6” or “Teric BL8”.

Preferably the surfactant is present in the solid substantially water-soluble composition in an amount of less than 25%. More preferably the surfactant is present in an amount of less than 15% of the composition. In a preferred form of the invention, the surfactant is present in an amount of about 10% of the composition. In a further form of the invention, the surfactant is present in an amount of less than 10% of the composition.

The surfactant is preferably a liquid or a blend of liquids. The surfactant preferably has a viscosity of less than about 400 mPa·s at 20 degrees Celsius. More preferably the viscosity is less than about 100 mPa·s at 20 degrees Celsius. In a preferred form of the invention, the viscosity is less than 70 mPa·s at 20 degrees Celsius.

The present invention also provides a method for the preparation of a solid substantially water-soluble adjuvant composition comprising:
(i) milling a particulate nitrogen-based plant nutrient; and
(ii) mixing an acidifier with the milled nitrogen-based plant nutrient so that the acidifier is supported on the milled nitrogen-based plant nutrient.

The acidifier may take various forms and may comprise one or more compounds selected from the group comprising inorganic liquid acids, inorganic solid acids, organic liquid acids, and organic solid acids. Preferably the acidifier is water soluble. Preferably the acidifier comprises one or more compounds selected from the group comprising acetic acid, propionic acid, phosphoric acid, and citric acid.

The invention still further provides a method of controlling weeds comprising forming an aqueous mixture of a solid substantially water-soluble adjuvant composition as herein described with a herbicide and applying the mixture to the weeds to be controlled. Preferably the aqueous mixture is applied by spraying onto the weeds.

Thus the inventors have found that by appropriate size reduction pre-treatment of the particulate nitrogen-based plant nutrient such as ammonium sulfate, the surfactant and/or acidifier may be absorbed and/or adsorbed onto the particles of the ammonium sulfate. Furthermore the choice of a surfactant, an acidifier or an acidifier/surfactant blend of appropriate viscosity leads to an improved product which displays more rapid dissolution into water. In this way an adjuvant composition is formed which retains the separate properties of the components but also has the benefit of being free-flowing and therefore easy to package, as well as having suitable dissolvability in water.

The word “solid” in the specification is to be understood to mean a material that substantially keeps its shape, that is one that is not a liquid or a gas.

Throughout the specification, unless the context requires otherwise, the word “comprise” or variations such as “comprises” or “comprising”, will be understood to imply the inclusion of a stated integer or group of integers but not the exclusion of any other integer or group of integers. Likewise the word “preferably” or variations such as “preferred”, will be understood to imply that a stated integer or group of integers is desirable but not essential to the working of the invention.

### BRIEF DESCRIPTION OF THE DRAWINGS

The nature of the invention will be better understood from the following detailed description of several specific embodiments of the solid substantially water-soluble adjuvant composition and the method of the invention, given by way of example only, with reference to the accompanying drawing, in which:

FIG. 1 shows a flow diagram of an embodiment of the method of the present invention.

### DETAILED DESCRIPTION OF PREFERRED EMBODIMENTS

The nature of the invention will be better understood from the following detailed description of several specific embodiments of the invention, given by way of example only, with reference to the accompanying examples.

The following materials were used in the examples of the invention given below.

Materials

| | | |
|---|---|---|
| Teric N8 | Liquid surfactant | Nonyl phenol ethoxylated with 8 moles |
| | Wetting agent | of ethylene oxide (Huntsman Corp. |
| | | Aust. P/L) - Viscosity 350 |
| | | mPa · s @ 20° C. |
| Teric 9A6 | Liquid surfactant | C9-C12 alcohol ethoxylated with 6 |
| | Wetting agent | moles of ethylene oxide (Huntsman |
| | | Corp. Aust. P/L) - Viscosity |
| | | 38 mPa · s @ 20° C. |
| Teric BL8 | Liquid surfactant | Modified alcohol alkoxylate, |
| | Wetting agent | (Huntsman Corp. Aust. P/L) - |
| | | Viscosity 63 mPa · s @ 20° C. |
| BDI | Inert diluent | Diethyl glycol monobutyl ether |
| SEA-39 | Antifoam agent | Organosiloxane antifoam (Wacker |
| | | Chemicals Aust. P/L). |
| Urea | Fertiliser carrier | Crystalline |
| | (nitrogen-based | |
| | plant nutrient) | |
| Ammonium | Fertiliser carrier | Crystalline technical grade |
| Sulfate | (nitrogen-based | |
| | plant nutrient) | |
| Propionic | Liquid acidifier | Liquid carboxylic acid |
| acid | | |
| Citric acid | Solid acidifier | Solid carboxylic acid |

Particle Size Range of ammonium sulfate is given below, for the as received ammonium sulfate, as well as the milled and sieved ammonium sulfate.

| Size Range | As Received | Milled | Sieved |
|---|---|---|---|
| % Pass 2.00 mm | 100.0 | 100.0 | 100.0 |
| % Pass 1.00 mm | 84.0 | 100.0 | 100.0 |
| % Pass 0.50 mm | 28.0 | 78.0 | 100.0 |
| % Pass 0.25 mm | 1.0 | 38.0 | 100.0 |

Methods

The following methods were used to assess formulations prepared in the examples of the invention given in the specification.

Flowability was assessed in the examples by using the following test:

Sufficient ammonium sulfate was added to a 50 mL glass beaker to give 20.0 g of final product (adjuvant composition). The surfactant (liquid adjuvant component) was added and the mixture stirred thoroughly by hand using a metal spatula for two minutes to combine, taking particular care to scrape the sides of the beaker clean. The resulting mixture was then settled by tapping the beaker sharply on a hard surface 10 times. The beaker was then slowly upended through 90 degrees to allow the material to drop out. The upended beaker was then allowed to fall from a height of 25 mm to dislodge any loosely adhering material. The beaker and residue was then weighed and the percentage of material remaining adhered to the surface of the beaker determined. The percent flowability was then calculated as 100 minus the percentage of sample that remained in the beaker. It is desirable for the flowability thus calculated to be as high as possible (maximum of 100% equals no residue remaining). Formulations that achieved a flowability of >98% in this test were observed in practice to be substantially free flowing and left no significant residue in the packaging (GOOD flowability).

Dissolvability was assessed in the examples by the following test:

To a 250 mL measuring cylinder containing 200 mL of tap water was added 2.0 g of product (adjuvant composition). The measuring cylinder was then stoppered and the number of inversions needed to mix and dissolve the sample determined by upending the cylinder through 90 degrees and back again with each cycle taking about 2 seconds. It is desirable for the dissolvability thus calculated to be as low as possible. Formulations that achieved a dissolvability of 6 revolutions or less were observed in practice to dissolve very quickly.

Foam height was determined by CIPAC MT-47 (2.0 g of adjuvant composition) added to 200 mL of tap water in a 250 mL measuring cylinder, cylinder inverted 30 times and height of foam thus generated measured).

Wettability was determined by CIPAC MT-53 by adding sufficient adjuvant composition to water to give 0.1% wetter in solution. The mixture was stirred for 5 minutes and the time taken for a strip of standard cotton tape to wet was determined.

Solution pH (acidity/alkalinity) was determined by instrumental measurement of the solution prepared for measurement of wettability described above.

Except where otherwise indicated, the nitrogen based plant nutrient used was ammonium sulfate (AMS) milled in one of two ways (1) small scale (about 80 g) batch-wise under dry conditions for 30 seconds in a coffee grinder and (2) large scale using a 30 cm diameter ICAL hammer mill running at 2,900 rpm fitted with a 4 mm screen and feed screw set to give a throughput of about 1,500 kg/hour. For large scale milling, silica anti-caking agent was pre-blended into the crystalline AMS at a level of 0.25% prior to milling.

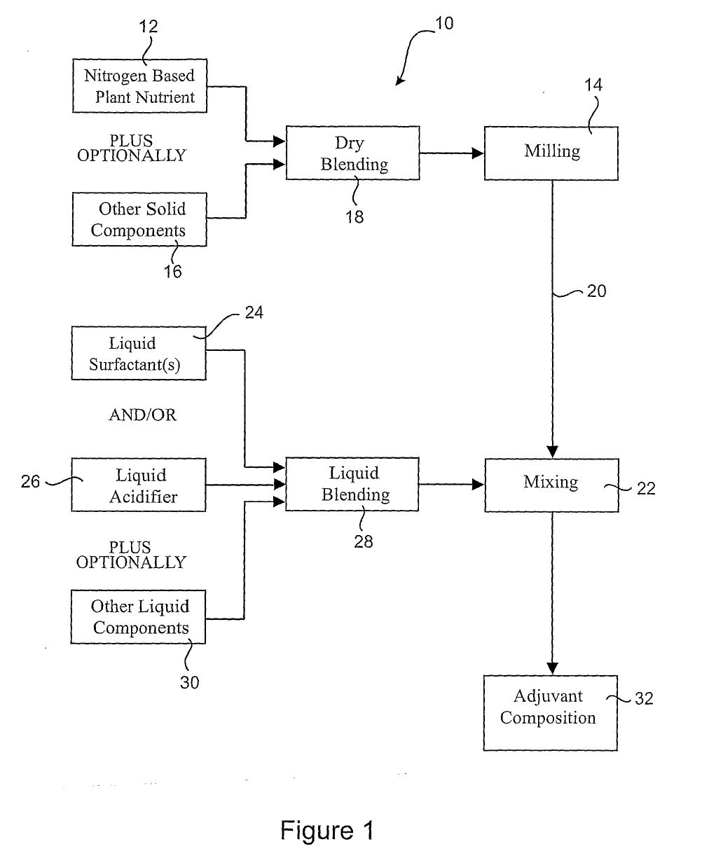

For ease of understanding of the invention, a schematic representation of the various steps in an embodiment of the method of the invention is shown in FIG. 1.

A method 10 for the preparation of a solid substantially water-soluble adjuvant composition 32 comprises the step of milling 14 a particulate nitrogen-based plant nutrient 12. Prior to the milling step 14, other solid components 16 such as anti-caking agents may be added to the nitrogen-based plant nutrient in a dry blending step 18.

The milled nitrogen-based plant nutrient 20 (optionally with other solid components) is then passed to a mixing step 22. The mixing step typically takes place after stirring for about 5 minutes at room temperature. During mixing a liquid surfactant 24 is added to the milled nitrogen-based plant nutrient 20 to produce the adjuvant composition 32 in which the surfactant 24 is supported on the milled nitrogen-based plant nutrient 20.

As an alternative, an acidifier 26 (either a liquid or a solid acidifier) is added to the milled nitrogen-based plant nutrient 20 so that the acidifier 26 is supported on the milled nitrogen-based plant nutrient 20. In a further alternative form of the invention, a liquid surfactant 24 is blended with a liquid acidifier 26 in a liquid blending step 28. The liquid blended composition is then added to the milled nitrogen-based plant nutrient 20 in the mixing step 22 as shown in FIG. 1. Other liquid components 30 such as antifoaming agents may also be added in the liquid blending step 28.

### EXAMPLES

Several examples of a solid substantially water-soluble adjuvant composition and a method of preparing same will now be described in detail.

Example 1 shows the mixing of untreated as-received ammonium sulfate with liquid adjuvants (surfactants and acidifiers)

Example 2 shows the addition of a small amount of anti-caking agent to the unmilled as-received ammonium sulfate.

Examples 3 and 4 show the mixing of milled ammonium sulfate with liquid adjuvants (surfactants). Example 4 is given as a Reference Composition or Formulation.

Example 5 shows the mixing of milled ammonium sulfate with liquid adjuvants (acidifiers).

Example 6 shows the mixing of liquid adjuvant (surfactant) with ammonium sulfate of different particle size distributions.

Example 7 shows the mixing of milled ammonium sulfate with liquid adjuvants of varying viscosity.

Example 8 shows the effects of ageing the composition of Example 4.

Example 9 describes the addition of an antifoaming agent to improve foaming behaviour of high foaming surfactants.

Example 10 illustrates the use of urea as the nitrogen based plant nutrient.

Example 11 shows the mixing of milled ammonium sulfate with blends of liquid propionic acid acidifier plus low and high viscosity liquid surfactants.

Example 12 shows the mixing of milled ammonium sulfate plus solid citric acid acidifier with a low viscosity liquid surfactant.

Example 13 describes the use of the reference composition of Example 4 by mixing with the herbicide glyphosate in a farm spray tank and application to an agricultural situation.

### Example 1

This example illustrates the poor handling characteristics (flowability) and poor dissolvability of a simple blend of crystalline technical ammonium sulphate (unmilled) and liquid adjuvant (surfactant or acidifier).

Crystalline technical ammonium sulfate (AMS) as received was mixed with Teric N8 surfactant or Teric BL8 surfactant or propionic acid acidifier as follows:

(a) Viscous Surfactant (Teric N8)

| | |
|---|---|
| Coarse Crystalline AMS | 90.0% w/w (No anti-caking agent used) |
| Teric N8 liquid surfactant | 10.0% w/w, Viscosity 350 mPa · s @ 20° C. |

| | | | |
|---|---|---|---|
| Flowability: | 86.7% | POOR | Large amount of material |
| | | | adhering to container |
| Dissolvability: | 8 revolutions | SLOW | Dispersed/dissolved slowly |

Flowability was poor (sample remained wet and sticky). Although dissolvability into water was complete, the rate was slow.

(b) Non-Viscous Surfactant (Teric BL8)

| | |
|---|---|
| Coarse Crystalline AMS | 90.0% w/w (No anti-caking agent used) |
| Teric BL8 liquid surfactant | 10.0% w/w, Viscosity 62 mPa · s @ 20° C. |

| | | | |
|---|---|---|---|
| Flowability: | 90.2% | POOR | Large amount of material |
| | | | adhering to container |
| Dissolvability: | 5 revolutions | MEDIUM | Dispersed/dissolved OK |

Flowability was poor (sample remained wet and sticky). Although dissolvability into water was complete, the rate was moderately slow.

(c) Liquid Acidifier (Propionic Acid)

| Coarse Crystalline AMS | | 90.0% w/w (No anti-caking agent used) | |
|---|---|---|---|
| Propionic Acid Liquid Acidifier | | 10.0% w/w, Viscosity 1.2 mPa · s @ | |
| | | 20° C. | |
| Flowability: | 94.0% | POOR | Large amount of material |
| | | | adhering to container |
| Dissolvability: | 5 revolutions | MEDIUM | Dispersed/dissolved OK |

Flowability was poor (sample remained wet and sticky). Although dissolvability into water was complete, the rate was moderately slow.

### Example 2

This example illustrates how admixing a small amount of anti-caking agent with unmilled AMS does not improve handling characteristics (flowability)

Crystalline technical ammonium sulfate (AMS) as received was mixed with 0.25% anti-caking agent and the blend mixed with Teric BL8 surfactant as follows:

| | |
|---|---|
| Coarse Crystalline AMS | 90.0% w/w (Anti-caking agent used) |
| Teric BL8 liquid surfactant | 10.0% w/w, Viscosity 62 mPa · s @ 20° C. |

| | | | |
|---|---|---|---|
| Flowability: | 93.1% | POOR | Large amount of material |
| | | | adhering to container |
| Dissolvability: | 6 revolutions | MEDIUM | Dispersed/dissolved OK |

The result was the same as Example 1. Addition of anticaking agent (0.25%, sufficient to aid milling) did not improve product handling properties.

### Example 3

This example illustrates how handling characteristics (Flowability) may be enhanced by milling the crystalline ammonium sulfate.

| | |
|---|---|
| Milled AMS | 90.0% w/w (Anti-caking agent used) |
| Teric N8 liquid surfactant | 10.0% w/w, Viscosity 350 mPa · s @ 20° C. |

| | | | |
|---|---|---|---|
| Flowability: | 99.3% | GOOD | Virtually no residue |
| Dissolvability: | 12 revolutions | SLOW | Dispersed/dissolved slowly |

Although flowability was good, surprisingly dissolvability remained slow. The same result was achieved when milled AMS with no anti-caking agent present was used. The unexpectedly slow dissolvability of this product despite containing the milled ammonium sulfate led the inventors to believe that the use of a surfactant with a high viscosity may be responsible for the slow dissolvability. Steps were then taken to use surfactants or blends with a lower viscosity.

### Example 4

This example illustrates the preparation and evaluation of a flowable adjuvant powder formulation of ammonium sulfate and liquid adjuvant of lower viscosity in accordance with the present invention. This formulation was designated as the Reference Formulation and was compared with other formulations in later examples.

| | |
|---|---|
| Milled AMS | 90.0% w/w (Anti-caking agent used) |
| Teric BL8 liquid surfactant | 10.0% w/w, Viscosity 62 mPa · s @ 20° C. |

| | | | |
|---|---|---|---|
| Flowability: | 99.4% | GOOD | Virtually no residue |
| Dissolvability: | 4 revolutions | FAST | Dispersed/dissolved quickly |
| Wettability | 4 seconds | GOOD | Wetted quickly |

Flowability, dissolvability and wettability were very good. The same result was achieved when milled AMS with no anti-caking agent present was used.

### Example 5

This example illustrates the preparation and evaluation of a flowable adjuvant powder formulation of ammonium sulfate and acidifier in accordance with the present invention.

| | |
|---|---|
| Milled AMS | 90.0% w/w (Anti-caking agent used) |
| Propionic Acid Liquid | 10.0% w/w, Viscosity 5 mPa · s @ 20° C. |
| Acidifier | |

| | | | |
|---|---|---|---|
| Flowability: | 99.0% | GOOD | Virtually no residue |
| Dissolvability: | 4 revolutions | FAST | Dispersed/dissolved quickly |

Both flowability and dissolvability were very good.

### Example 6

This example illustrates the relationship between liquid adjuvant loading and ammonium sulfate particle size distribution on formulation flowability.

| | |
|---|---|
| Surfactant | Flowability (%) |

| Loading | Unmilled | Milled | 100% |
|---|---|---|---|
| BL8 % w/w | AMS | AMS | −250 um AMS |
| 5 | 94.4 - FAIR | | |
| 10 | 89.7 - POOR | 99.7 - GOOD | |
| 15 | | 97.5 - FAIR | |
| 20 | | 97.1 - FAIR | 99.0 - GOOD |
| 25 | | 1.0 - VERY | 97.0 - FAIR |
| | | POOR | |

As the liquid adjuvant loading was increased, good flowability could be maintained by reducing the AMS particle size. As shown in the table, with a surfactant loading of 20%, by using 100% milled 250 um AMS, the flowability was improved from 97.1% to 99%. With a surfactant loading of 25%, by using 100% milled 250 um AMS, the flowability was improved from 1% to 97%.

### Example 7

This example illustrates the impact of liquid adjuvant viscosity characteristics on product flowability and dissolvability:

Samples were made using milled ammonium sulfate (about 0.25% anti-caking agent present) with 10% liquid surfactant loading.

| | Surfactant | | |
|---|---|---|---|
| | Blend | | Product |
| | Viscosity | Product | Dissolvability |
| Liquid Surfactant | (mPa · s) | Flowability (%) | (revs) |
| 100% N8 | 350 | 99.6 - GOOD | 12 - VERY |
| | | | SLOW |
| 75% N8 + 25% 9A6 | 174 | 99.5 - GOOD | 9 - SLOW |
| 50% N8 + 50% 9A6 | 110 | 99.7 - GOOD | 9 - SLOW |
| 25% N8 + 75% 9A6 | 62 | 99.6 - GOOD | 4 - FAST |
| 100% 9A6 | 44 | 99.5 - GOOD | 2 - FAST |
| 25% N8 + 75% BDI | 13 | 99.1 - GOOD | 2 - FAST |
| 50% N8 + 50% BDI | 32 | 99.2 - GOOD | 3 - FAST |
| 75% N8 + 25% BDI | 88 | 98.8 - GOOD | 2 - FAST |

It can be seen from the table that the flowability and dissolvability of the product was the most favourable for surfactants and surfactant blends of lower viscosity, preferably of viscosity of about 100 mPa·s or less.

### Example 8

This example illustrates the stability of a flowable adjuvant powder formulation of ammonium sulfate and surfactant prepared in accordance with the present invention. Reference Formulation prepared as per example 4 was subjected to the following environmental conditions to simulate accelerated ageing: (1) heated in an oven set at 54° C. for two weeks (2) cooled in a refrigerator set at −4 degrees Celsius for two weeks (3) subjected to a pressure of 110 g/sq·cm at ambient temperature for two weeks.

Results:

No physical changes were observed to occur. No separation of liquid was observed to occur.

| | | Dissolvability | Wettability |
|---|---|---|---|
| Storage Regime | Flowability (%) | (revs) | (sec) |
| (1) Ambient | 99.9 - GOOD | 4 - FAST | 5 - GOOD |
| (2) 54° C. | 99.9 - GOOD | 5 - FAST | 4 - GOOD |
| (3) −4° C. | 99.9 - GOOD | 4 - FAST | 4 - GOOD |
| (4) Pressure Test | 99.9 - GOOD | 4 - FAST | 5 - GOOD |

### Example 9

This example illustrates the addition of an antifoaming agent to improve foaming behaviour of high foaming surfactants.

| Parameter | No Antifoam | | Antifoam Present | |
|---|---|---|---|---|
| Milled AMS | 90.0% | | 90.0% | |
| Teric 9A6 Liquid Surfactant | 10.0% | | 10.0% | |
| SEA 39 Antifoam Dispersion | 0.00% | | 0.08% | |
| Flowability (%) | 99.5 - GOOD | | 99.0 - GOOD | |
| Dissolvability (revs) | 2 - FAST | | 2 - FAST | |
| Wettability (sec) | 2 - GOOD | | 2 - GOOD | |
| | Foam Height | | Foam Height | |
| Initial | 80 | mm | 61 | mm |
| 1 minute | 75 | | 18 | |
| 3 minutes | 75 | | 9 | |
| 5 minutes | 65 | | 8 | |

### Example 10

This example illustrates the use of urea as the nitrogen based plant nutrient carrier.

| | |
|---|---|
| Milled Urea | 90.0% w/w |
| Teric BL8 liquid surfactant | 10.0% w/w |

| | | | |
|---|---|---|---|
| Flowability: | 99.9% | GOOD | Virtually no residue |
| Dissolvability: | 3 revolutions | FAST | Dispersed/dissolved quickly |
| Wettability | 4 seconds | GOOD | Wetted very quickly |

Performance was equivalent to the Reference Formulation (Teric BL8 blended with milled ammonium sulfate—refer to example 4).

### Example 11

This example illustrates the preparation and evaluation of tri-function flowable adjuvant compositions of ammonium sulfate, a surfactant and a liquid acidifier in accordance with the present invention.

(a) Using Viscous Surfactant (Teric N8)

| | | | |
|---|---|---|---|
| Milled AMS | 90.0% w/w | | (No anti-caking agent used) |
| Teric N8 liquid | 5.0% w/w | | |
| surfactant* | | | *Preblended: Blend Viscosity |
| Propionic acid liquid | 5.0% w/w | {close oversize brace} | 21 mPa · s @ 20° C. |
| acidifier* | | | |

| | | | |
|---|---|---|---|
| Flowability: | 98.8% | GOOD | Virtually no residue |
| Dissolvability: | 4 revolutions | FAST | Dispersed/dissolved |
| | | | quickly |
| Wettability | 6 seconds | GOOD | Wetted quickly |
| pH Water Only: | 7.3 | Adjuvant | 4.0 |
| | | Solution (2%): | |

This example also illustrates how the acidifier has acted to reduce the viscosity of the liquid blend resulting in good dissolvability (compare with Example 3 containing only Teric N8 and Example 7, viscosity series). The solution pH was reduced to below 6 as desired to assist in improving pesticide penetration and reducing decomposition of the pesticide.

(b) Using Non-Viscous Surfactant (Teric BL8)

| | | | |
|---|---|---|---|
| Milled AMS | 90.0% w/w | | (No anti-caking agent used) |
| Teric BL8 liquid | 5.0% w/w | | |
| surfactant | | | Preblended: Blend Viscosity |
| Propionic acid liquid | 5.0% w/w | {close oversize brace} | 15 mPa · s @ 20° C. |
| acidifier | | | |

| | | | |
|---|---|---|---|
| Flowability: | 98.9% | GOOD | Virtually no residue |
| Dissolvability: | 4 revolutions | FAST | Dispersed/dissolved |
| | | | quickly |
| Wettability | 12 seconds | GOOD | Wetted quickly |
| pH Water Only: | 7.3 | Adjuvant | 4.1 |
| | | Solution (2%): | |

### Example 12

This example illustrates the preparation and evaluation of a tri-function flowable adjuvant powder formulation of ammonium sulfate, surfactant and solid acidifier in accordance with the present invention.

| | | | |
|---|---|---|---|
| Milled AMS* | 80.0% w/w | | |
| | | {close oversize brace} | *Preblended prior to |
| Citric Acid solid acidifier* | 10.0% w/w | | milling |
| Teric BL8 liquid surfactant | 10.0% w/w | | |

| | | | |
|---|---|---|---|
| Flowability: | 99.5% | GOOD | Virtually no residue |
| Dissolvability: | 4 revolutions | FAST | Dispersed/dissolved |
| | | | quickly |
| Wettability | 3 seconds | GOOD | Wetted quickly |
| pH Water Only: | 7.3 | Adjuvant | 3.2 |
| | | Solution (1%): | |

Flowability, Dissolvability and Wettability were all very good. The solution pH was reduced to below 6 as desired.

### Example 13

This example demonstrates that formulations of the present invention have equivalent bioactivity when compared to separate commercially available ammonium sulfate and surfactant adjuvant formulations.

Reference Formulation of Example 4 was added to a farm spray tank along with the herbicide glyphosate to give a glyphosate/ammonium sulfate/surfactant (wetter) spray composition as recommended on the glyphosate directions for use label. The mixture was applied by spraying onto a weedy field comprising mainly volunteer wheat and stubble. Weed control was observed to be equivalent to adjacent fields sprayed using an equivalent spray mixture of conventional ammonium sulfate and wetter formulations.

In another evaluation, Reference adjuvant composition of Example 4 was mixed in a spray tank with the herbicide glyphosate and sprayed onto test weed plots. Weed control was compared to similar plots sprayed with (1) glyphosate only, (2) glyphosate+wetting agent, (3) glyphosate+ammonium sulfate and (4) glyphosate+separate formulations of wetting agent and ammonium sulfate. Weed control was observed to be better than glyphosate alone and equivalent to the glyphosate+separate formulations of wetting agent and ammonium sulfate (treatment 4).

A number of observations can be made from the Examples of the present invention as described above.

Example 1 show that the simple mixing of untreated as received ammonium sulfate with a liquid adjuvant component (viscous or non-viscous surfactant or acidifier) produces a mixture with poor flowability.

Example 2 shows that the addition of a small amount of anti-caking agent to the composition of Example 1(b) does not improve formulation flowability.

Examples 3, 4 and 5 show how milling the ammonium sulfate produces formulations with good flowability. By milling or sizing the ammonium sulfate to produce a particle size predominately below 500 micrometres, a product with 10% loading of liquid adjuvant can be made which satisfies preferred flowability criterion.

Example 6 shows the relationship between ammonium sulfate particle size and liquid loading, and illustrates how increasingly higher loadings of liquid adjuvant can be achieved by further reducing the particle size. By sizing the ammonium sulfate to a particle size of less than 250 microns, loadings of up to 20% can be achieved with good flowability as defined herein.

Viscous water soluble surfactants can display a tendency to gel when added to water. This is a disadvantage as it slows dissolution, particularly at low temperatures. Such surfactants when coated onto finely divided ammonium sulfate might be expected to display improved dissolution but surprisingly this was not found to be the case. Example 7 illustrates that high liquid adjuvant viscosity inhibited rapid dissolvability and that low viscosity liquids or blends with low viscosity (below about 100 mPa·s) displayed very good dissolvability. Any suitable surfactant may be used as the surface active component in accordance with this invention provided the viscosity requirement can be met. As illustrated in Example 7, the liquid component may be a single surfactant, a blend of surfactants, or a blend of surfactant and inert diluent. As illustrated in Example 11, a liquid acidifier may also perform this viscosity reduction role.

Long term stability of any formulation is necessary to ensure the product has adequate shelf life and will perform as expected. To simulate accelerated ageing, Reference Formulation (Example 4) was subjected to various extreme storage regimes. Performance with respect to flowability, dissolvability and wettability remained unchanged. This is illustrated in Example 8.

Optionally, other additives may be included in the formulation to impart other desirable characteristics. A compatible additive is an additive which when added to a formulation in accordance with the present invention does not result in the formulation failing to meet the flowability or dissolvability criteria defined herein. The compatibility of an additive may be readily tested by including the additive in the Reference Formulation (Example 4) and determining whether the resultant formulation meets both the flowability and dissolvability criteria.

When milling ammonium sulfate, it is desirable to add a free flowing agent (anti-caking agent) particularly in humid conditions to avoid mill blockages and handling difficulties caused by hardening of the milled material. A wide range of synthetic precipitated silicas, kaolins and attapulgites are available which can perform this task. Typically these synthetic precipitated materials are added at a level of about 0.05 to 5%. The inclusion of these materials does not impact negatively on the composition performance.

It is desirable to include a defoaming agent as many surfactants suitable as wetting agents also display a high propensity to produce foam during mixing in spray tank water. The defoaming agent may be chosen from any of the materials or blends of materials commonly used to effect defoaming, providing that the previously mentioned compatibility requirement is satisfied. In particular, it has been found that consistently good results are given by organosiloxane emulsions which are conveniently delivered by predispersion into the liquid surfactant prior to blending with the ammonium sulfate. Defoaming agents are widely used as a separately added spray tank component as mixtures with surfactants are invariably unstable resulting in phase separation of the surfactant and defoamer. This phase separation does not occur in compositions of the present invention. Example 9 illustrates the positive impact of added antifoaming agent when used with Teric 9A6. This surfactant has desirable wetting characteristics but a high propensity to foam.

The scope of this invention is not restricted to the use of ammonium sulfate as the nitrogen-based plant nutrient (carrier). Example 10 illustrates that urea may also be used as a carrier to give a combination formulation with surfactant that also has good flowability and dissolvability properties.

Examples 11 and 12 illustrate how the process can be extended to introduce an acidifier as a third component of the adjuvant composition. Any suitable acid may be added as an acidifier including a liquid or solid acid. Use of a liquid acidifier can have the advantage of viscosity reduction if the liquid surfactant has high viscosity, as illustrated in example 11. The use of a solid acidifier is illustrated in example 12. In this way a single adjuvant formulation with tri-functionality can be made, that is achieving the functional benefits of a nitrogen-based plant nutrient, a surfactant and an acidifier.

It can be seen that the present invention provides a method for reducing glyphosate tank mix antagonism using a single adjuvant composition comprising a blend of ammonium sulfate and surfactant added to a spray tank that replaces two separate adjuvant formulations as illustrated by Example 13.

Furthermore, it has been found that the formulations of this invention have adjuvancy equivalent to separate commercially available liquid and solid formulations of wetting agent (surfactant) and ammonium sulphate, typically combined in a spray tank at the point of use.

An important aspect of any combined adjuvant composition is the ratio of combined adjuvant components. This ratio should be consistent with the use rates of the separate components for the combined formulation to have maximum utility. Ammonium sulfate is typically used at a spray solution concentration of 0.5 to 2%. Wetting agents (surfactants) are typically used at a spray solution concentration of 0.05 to 0.2%. Thus a combined ammonium sulfate plus wetter adjuvant will ideally have the components present at a ratio in the range of 1 part wetter (surfactant) with 9 parts ammonium sulfate to 2 parts wetter (surfactant) with 8 parts ammonium sulfate. Combined adjuvant compositions made according to the present invention have a wetter (surfactant) to ammonium sulfate ratio in this biologically useful range, which makes them suitable as direct replacements for the currently used separate adjuvant formulations.

In summary, the present invention aims to provide a granular, solid adjuvant composition that is substantially water soluble and contains a nitrogen-based plant nutrient (such as ammonium sulphate) and a surfactant (or acidifier) in a biologically useful ratio. There are three main desirable requirements for the adjuvant composition of the invention, namely a high loading of surfactant, rapid and complete dissolution in water, and satisfactory product handling characteristics. These requirements mean that the adjuvant composition can be dissolved and/or dispersed in a spray tank suitable for use in an agricultural chemical application. The optimum adjuvant composition of the present invention is achieved by a combination of suitable particle size of the nitrogen-based plant nutrient and by choice of correct surfactant (or acidifier) viscosity.

Surprisingly, the formulations of this invention are substantially dust free, flowable, and dissolvable. Du e to the formulation being solid rather than liquid, the formulation can be inexpensively packaged into plastic bags. Rigid plastic packaging traditionally used to package conventional ammonium sulfate and surfactant adjuvant formulations generally weigh between 1000 and 1200 grams. Plastic bags suitable for packaging a product based on the present invention have a weight of typically about 100 to 120 grams. This represents a tenfold reduction in the weight of plastic packaging required and therefore contributes to a significant cost saving and reduced environmental impact of plastic waste.

Now that preferred embodiments of the solid substantially water-soluble adjuvant composition of the invention have been described in detail, it will be apparent that the composition provides a number of advantages over the prior art, including the following:

(i) The composition is an easy to handle solid granular material.

(ii) The composition contains no added water.

(iii) The composition contains generally more than 99% active adjuvant material.

(iv) The composition is generally more than 99% water soluble.

(v) The composition is substantially free-flowing and dissolves readily in water.

(vi) The composition may contain multiple adjuvant components (surfactant, acidifier and nitrogen-based plant nutrient) as a single formulation.

(vii) The composition usually contains the adjuvant components in a biologically useful ratio.

(viii) The composition can be made using a wide range of biologically useful components.

(ix) The composition may be easily packaged and stored; and,

(x) The composition is generally dust free thereby minimising the risk of health hazards.

1-54. (canceled)

55. A solid substantially water-soluble adjuvant composition comprising a liquid surfactant having a viscosity of less than 100 mPa·s at 20 degrees Celsius and particulate ammonium sulfate, the liquid surfactant being supported on the ammonium sulfate.

56. A solid substantially water-soluble adjuvant composition as defined in claim 55, wherein the liquid surfactant has a viscosity of less than 70 mPa·s at 20 degrees Celsius.

57. A solid substantially water-soluble adjuvant composition as defined in claim 55, wherein the particulate ammonium sulfate has been milled to a particle size of less than 1000 microns.

58. A solid substantially water-soluble adjuvant composition as defined in claim 57, wherein the particulate ammonium sulfate has been milled to a particle size of less than 500 microns.

59. A solid substantially water-soluble adjuvant composition as defined in claim 55, wherein the surfactant is a non-ionic, anionic or cationic surfactant.

60. A solid substantially water-soluble adjuvant composition as defined in claim 59, wherein the surfactant comprises one or more compounds selected from the group consisting of alkyl phenol ethoxylates, alkyl phenol alkoxylates, alcohol exthoxylates, alcohol alkoxylates, aminoethoxylates and modified alkylpolysiloxane surfactants.

61. A solid substantially water-soluble adjuvant composition as defined in claim 55, wherein the surfactant is present in the composition in an amount of less than 25% of the composition.

62. A solid substantially water-soluble adjuvant composition as defined in claim 55, wherein the solid substantially water-soluble adjuvant composition is granular.

63. A solid substantially water-soluble adjuvant composition as defined in claim 62, wherein the granules have a particle size of less than 3000 microns.

64. A solid substantially water-soluble adjuvant composition as defined in claim 57, wherein an anti-caking agent is added to the particulate ammonium sulfate prior to milling.

65. A solid substantially water-soluble adjuvant composition as defined in claim 55, wherein the solid substantially water-soluble adjuvant composition further comprises an acidifier.

66. A solid substantially water-soluble adjuvant composition as defined in claim 65, wherein the acidifier comprises one or more compounds selected from the group consisting of inorganic liquid acids, inorganic solid acids, organic liquid acids, and organic solid acids.

67. A solid substantially water-soluble adjuvant composition as defined in claim 65, wherein the acidifier has a viscosity of less than 400 mPa·s at 20 degrees Celsius.

68. A method for the preparation of a solid substantially water-soluble adjuvant composition comprising:
ii) milling particulate ammonium sulfate; and
(i) mixing a liquid surfactant having a viscosity of less than 100 mPa·s at 20 degrees Celsius with the particulate ammonium sulfate so that the liquid surfactant is supported on the milled ammonium sulfate.

69. A method for the preparation of a solid substantially water-soluble adjuvant composition as defined in claim 68, wherein the liquid surfactant has a viscosity of a viscosity of less than 70 mPa·s at 20 degrees Celsius.

70. A method for the preparation of a solid substantially water-soluble adjuvant composition as defined in claim 68, wherein the particulate ammonium sulfate is milled to a particle size distribution of 80% less than 1000 microns.

71. A method for the preparation of a solid substantially water-soluble adjuvant composition as defined in claim 70, wherein the particulate ammonium sulfate is milled to a particle size of less than 250 microns.

72. A method for the preparation of a solid substantially water-soluble adjuvant composition comprising:
ii) milling particulate ammonium sulfate; and
ii) mixing a liquid surfactant blend, the liquid surfactant blend having a viscosity of less than 100 mPa·s at 20 degrees Celsius, with the milled particulate ammonium sulfate so that the liquid surfactant blend is supported on the milled ammonium sulfate.

73. A method for the preparation of a solid substantially water-soluble adjuvant composition as defined in claim 72, wherein the liquid surfactant blend comprises one or more liquid surfactants.

74. A method for the preparation of a solid substantially water-soluble adjuvant composition as defined in claim 72, wherein the liquid surfactant blend further comprises a diluent.
